(19) United States
(12) Patent Application Publication
(10) Pub. No.: US 2009/0130759 A1
(43) Pub. Date: May 21, 2009

(54) Culture Medium Containing Kinase Inhibitor, and Use Thereof

(75) Inventors: Austin Gerard Smith, Cambridge (GB); Qi-Long Ying, Los Angeles, CA (US)

Correspondence Address: FINNEGAN, HENDERSON, FARABOW, GARRETT & DUNNER;LLP, 901 NEW YORK AVENUE, NW, WASHINGTON, DC, 20001-4413, US

(73) Assignee: The University Court of the University of Edinburgh, Edinburgh (GB)

(21) Appl. No.: 11/909,545

(22) PCT Filed: Mar. 22, 2006
(86) PCT No.: PCT/GB2006/001064

§ 371 Date: Dec. 20, 2007

(30) Foreign Application Priority Data
Mar. 23, 2005 (GB) 0505970.4

(51) Int. Cl. C12N 5/00; C12N 15/87

(52) U.S. Cl. 435/455; 435/404; 435/354; 435/366

(57) ABSTRACT

Pluripotent cells are maintained in a self-renewing state in serum-free culture medium comprising a gp130 agonist (LIF) and a GSK3 inhibitor.

### INTRODUCTION

The present invention relates to maintenance of a self renewing phenotype in pluripotent stem cells. The methods and compositions provided are suitable for culturing and isolating pluripotent stem cells such as embryonic stem (ES) cells, especially mammalian, including mouse and human, stem cells. In particular this invention relates to self-renewing cultures of mouse and human ES cells and to methods and compositions therefor.

### BACKGROUND

The establishment and maintenance of in vitro pluripotent stem cell cultures in the presence of medium containing serum and Leukaemia Inhibitory Factor (LIF) is well known (Smith et al. (1988) Nature 336: 688-90). Such methods have been used to maintain pluripotent embryonic stem (ES) cells from strains of “permissive” mice over many passages. Maintenance and self renewal of pluripotent stem cell cultures is further supported where the stem cells are cultured in the presence of feeder cells or extracts thereof, usually mouse fibroblast cells. Under such conditions it is possible to maintain human ES cells in a pluripotent state over many passages in culture.

In many cases ES cells can only be maintained, or are best maintained, using medium that contains serum or serum extract, and hence is undefined, or using cell culture conditions that require the presence of other cells, such as the fibroblast feeder cells used to maintain human ES cells. But any undefined component, whether in the medium or produced by e.g. the feeder cells, potentially interferes with or hinders research into ES cell propagation and differentiation. This prevents development of good manufacturing practices for therapeutic and other applications of ES cells and their progeny. Some defined ES cell media are known but alternative and preferably improved defined media are needed.

In prior applications by the applicants, WO-A-03/095628 and a later as yet unpublished application, culturing pluripotent stem cells, such as ES cells, in serum-free media comprising (1) agonists of gp130 (e.g. LIF) and (2) agonists of the TGF-α superfamily (e.g. BMP4) or Id signalling pathways is used to promote self renewal of the stem cells for multiple passages. In the presence of gp130 signalling, an agonist of the TGF-β superfamily or the Id signalling pathway surprisingly provided a self renewal stimulus rather than a pro-differentiation signal. Nevertheless, ever improved efficiencies in maintaining pluripotent cells in a self renewing state and media for transferring pluripotent cells away from feeder cells or away from feeder-conditioned medium is desired.

Sato N, et al, Nat. Med. 2004, January 10(1) pp 55-63 describe the effects of a Glycogen Synthase Kinase 3 (GSK3) inhibitor, 6-bromoindirubin-3′-oxime, on mouse and human ES cells in serum containing medium. These effects, however, were observed only over a very short time frame, too short for firm conclusions to be drawn, and the influence of unknown factors in the undefined media used in that study may be significant. The inventors of the present invention have tried but failed to repeat the results, and have in fact found effects opposite to those described—see the comparative example below.

For preparation of ES cell culture media it is desired to provide individual media components in as pure a form as possible. However, most media components are cytokines the purity of which is compromised by the need to manufacture them in cellular systems and then remove potential contaminants from the production broth. Another problem with some cytokines is that they have a narrow range of concentration over which they are effective and non-toxic. Media components which have a broader range and/or are less toxic at higher concentrations would be highly useful. Cytokines can also have limited stability in storage, and more stable media components are sought.

An object of the invention is to overcome or at least ameliorate problems in the art, preferably to provide alternative, more preferably improved, methods of culturing and culture media suitable for pluripotent stem cells, which are capable of supporting self-renewal of said stem cells for many passages. A further object of the invention is to provide an alternative culturing system that permits maintenance of a pluripotent stem cell culture in vitro until differentiation of the cells can be induced in a controlled manner. A still further object of the invention is to provide methods and compositions that enhance the derivation and isolation of pluripotent stem cells and facilitate their derivation and isolation from organisms refractory to ES cell isolation or from which pluripotent stem cells have not yet been isolated.

### SUMMARY OF THE INVENTION

According to a first aspect of the present invention, inhibition of GSK3 in a pluripotent cell in the presence of gp130 signalling is used to promote self-renewal of the cell.

In accordance with the present invention, pluripotent stem cells, such as ES cells, are cultured in medium, preferably serum-free, comprising an agonist of the gp130 signalling pathway (e.g. LIF) coincident with inhibition of GSK3 (e.g. using a small molecule GSK3 inhibitor). Self renewal of the stem cells for multiple passages is thereby promoted. Hence, in the presence of gp130 signalling, inhibition of this gsk enzyme in the pluripotent cells provides a self renewal stimulus.

The invention has a number of applications. A combination of gp130 signalling and inhibition of GSK3 can be used to grow pluripotent cells, especially ES cells, and, where they have been derived or grown on feeders, to adapt pluripotent cells, especially ES cells, to grow without feeders. A method of expanding stem cells in culture comprises culturing the cells in the presence of an inhibitor of GSK3. Culture medium can be prepared containing one or more GSK3 inhibitors. ES cells can be derived using GSK3 inhibitors and gp130 agonists.

### DETAILED DESCRIPTION OF THE INVENTION

Reference to GSK3 inhibition refers to inhibition of one or more GSK3 enzymes. In specific embodiments GSK3-β is inhibited. GSK3-α inhibitors are also suitable, and in general inhibitors for use in the invention inhibit both. A wide range of GSK3 inhibitors are known and, by way of example, the inhibitors CHIR 98014, CHIR 99021, AR-AO144-18, SB216763 and SB415286 have been used to promote self renewal of ES cells. Other inhibitors are known and useful in the invention. The inhibitors of certain embodiments are specific for GSK3-β and GSK3-α, substantially do not inhibit erk2 and substantially do not inhibit cdc2. Preferably the inhibitors have at least 100 fold, more preferably at least 200 fold, very preferably at least 400 fold selectivity for human GSK3 over mouse erk2 and/or human cdc2, measured as ratio of IC₅₀ values; here, reference to GSK3 IC₅₀ values refers to the mean values for human GSK3-β and GSK3-α. Good results have been obtained with CHIR 99021 and CHIR 98014, which both are specific for GSK3. Examples of GSK3 inhibitors are described in Bennett C, et al, J. Biological Chemistry, vol. 277, no. 34, Aug. 23, 2002, pp 30998-31004 and in Ring D B, et al, Diabetes, vol. 52, March 2003, pp 588-595. Suitable concentrations for use of CHIR 99021 are in the range 0.01 to 100, preferably 0.1 to 10, more preferably 0.3 to 3 micromolar.

In examples below, we have cultured mouse ES cells in the presence of a GSK3 inhibitor together with gp130 signalling to promote self renewal. In other specific examples described below in more detail, a method of promoting self-renewal of mouse pluripotent cells in culture comprises (i) inhibiting GSK3, and (ii) activating gp130 downstream signalling.

Activation of one or more gp130 downstream signalling pathways can be achieved by use of a cytokine acting through gp130, for example a cytokine or other agonist of the LIF receptor. Cytokines capable of acting through gp130, and thus of activating gp130 signal transduction, include LIF, CNTF, cardiotrophin, oncostatin M, IL-6 plus sIL-6 receptor and hyper IL-6. Suitable cytokines include mimetics, fusion proteins or chimaeras that can bind to and/or activate signalling though gp130. The role of cytokines acting through gp130 in the presence of serum is well established, but the capacity of those cytokines to sustain undifferentiated cells in the absence of serum is limited.

An advantage of the invention is that in the presence of GSK3 inhibitor and gp130 agonist pluripotent cells can be grown in defined medium. The present invention therefore enables alternative and/or improved culture of ES cells in medium that is free of serum, serum extract, feeder cells and feeder cell extract.

Embryonic stem cells have been reported from a number of mammalian sources including mouse (Bradley et al (1984) Nature 309: 255-56), American mink (Mol Reprod Dev (1992) December; 33(4):418-31), pig and sheep (J Reprod Fertil Suppl (1991); 43:255-60), hamster (Dev Biol (1988) May; 127(1):224-7) and cow (Roux Arch Dev Biol (1992); 201: 134-141). Specific examples herein use mouse and human ES cells. It will be appreciated that the methods and compositions of the present invention are suitable for adaptation to culturing of other mammalian pluripotent cell cultures, thus including primate, especially human, rodent, especially mouse and rat, and avian pluripotent stem cells, especially ES cells.

A second aspect of the invention provides a method of culture of pluripotent cells, especially ES cells, so as to promote self renewal, comprising maintaining the cells in medium containing:—
(1) an inhibitor of GSK3; and
(2) an activator of a gp130 downstream signalling pathway.

Methods of the invention can be used generally for growing pluripotent cells, including growing ES cells in medium which is free of serum and free of serum extract, which cells have previously been passaged in the presence of serum or serum extract. Preferably, such methods are also carried out in the absence of feeder cells and/or feeder cell extracts. For example, culture of ES cells can be carried out comprising the steps of:—
maintaining the ES cells in a pluripotent state in culture, optionally on feeders, in the presence of a cytokine acting though gp130 and serum or an extract of serum;
passaging the ES cells at least once;
withdrawing the serum or the serum extract from the medium and withdrawing the feeders (if present), so that the medium is free of feeders, serum and serum extract; and
subsequently maintaining ES cells in a pluripotent state in the presence of an inhibitor of GSK3 and an activator of a gp130 downstream signalling pathway.

At around the time that the serum or extract of serum is withdrawn from the medium, it is an option to add to the medium an agent that suppresses differentiation, for example, an FGF-receptor inhibitor. It is an option for the inhibitor of differentiation to be withdrawn at the same time as or subsequent to maintenance of the cells in the presence of an Id protein. The serum or extract can be withdrawn at the same time as or before or after the feeder cells or extract is withdrawn.

The present invention also provides a method of obtaining a transfected population of ES cells, comprising:—
transfecting ES cells with a construct encoding a selectable marker;
plating the ES cells;
culturing the ES cells in the presence of an inhibitor of GSK3 and an activator of gp130 downstream signalling pathways; and
selecting for cells that express the selectable marker.

The selectable marker may encode antibiotic resistance, a cell surface marker or another selectable marker as described e.g. in EP-A-0695351, and preferably comprises a nucleotide sequence encoding the selectable marker operatively linked to a promoter which preferentially expresses the selectable marker in desired cells.

In a further embodiment, the present invention provides a method of culture of pluripotent, especially ES, cells, comprising the steps of transferring an individual cell to a culture vessel, such as an individual well on a plate, and culturing the cell in the presence of a GSK3 inhibitor and an activator of gp130 downstream signalling pathways, so as to obtain a clonal population of pluripotent, especially ES, cells, all of which are progeny of a single cell.

Once a stable, homogenous culture of ES cells is obtained, the culture conditions can be altered to direct differentiation of the cells into one or more cell types selected from ectodermal, mesodermal or endodermal cell fates. Addition of, or withdrawal of cytokines and signalling factors, can enable the derivation of specific differentiated cell populations at high efficiency. Differentiation of an ES cell towards a non-neuroectodermal fate may be achieved by maintaining the ES cell in the presence of a cytokine acting through gp130 and a GSK3 inhibitor and then withdrawing the cytokine whilst maintaining the GSK3 inhibitor and/or adding a further signalling molecule capable of directing differentiation. The methods described above all optionally includes the step of obtaining and/or isolating a differentiated cell which is the product of the process.

Further aspects of the invention provide for cell culture media. One medium is for self-renewal of pluripotent, especially ES, cells, the medium comprising an inhibitor of GSK3 and an activator of a gp130 downstream signalling pathway. Another medium of the invention is a stem cell culture medium, comprising an inhibitor of GSK3.

The invention provides medium that is free of serum and serum extract. One such medium comprises:—
basal medium;
a GSK3 inhibitor;
an activator of gp130 downstream signalling pathways; and
an iron-transporter;
   wherein the medium is optionally free of serum and serum extract.

Preferred medium for human pluripotent stem cells may be different in that it may be free of gp130 agonists; it hence comprises a GSK3 inhibitor, and an agonist of the FGF receptor, optionally supplemented with an activator of gp130 downstream signalling pathways. A specific human pluripotent stem cell medium comprises (a) a GSK3 inhibitor, and (b) FGF, and optionally (c) LIF or hyper IL-6. Preferred medium for pluripotent stem cells other than human stem cells, such as but not limited to medium for mouse cells, comprises a GSK3 inhibitor, an activator of gp130 downstream signaling pathways and an inhibitor of ES cell differentiation. A specific medium for non-human pluripotent stem cells comprises (a) LIF, (b) a GSK3 inhibitor and (c) optionally an inhibitor of FGF. Substitutions of media components can be made as described herein.

Basal medium is medium that supplies essential sources of carbon and/or vitamins and/or minerals for the cells. The basal medium is generally free of protein and incapable on its own of supporting self-renewal of cells. The iron transporter provides a source of iron or provides ability to take up iron from the culture medium. Suitable iron transporters include transferrin and apotransferrin. It is preferred that the medium further comprises one or more of insulin or insulin-like growth factor and albumin (preferably recombinant) or albumin substitute, and is free of feeder cells and feeder cell extract.

A particular medium of the invention comprises LIF, GSK3 inhibitor, insulin, albumin and transferrin, with or without additional basal medium. In this medium, LIF can be substituted by other activators of gp130 signalling, though preferred medium comprises the gp130 receptor binding cytokine, LIF, suitable concentrations of which are generally between 10 U/ml and 1000 U/ml, more preferably between 50 U/ml and 500 U/ml, even more preferably in the region of 100 U/ml. The GSK3 inhibitor is preferably as described herein in more detail.

The invention further provides a method of deriving a pluripotent cell from a blastocyst, comprising:—
(1) obtaining a blastocyst;
(2) culturing the blastocyst in the presence of an activator of gp130 downstream signalling, to obtain an inner cell mass;
(3) dissociating the inner cell mass;
(4) isolating a cell or cells from the dissociated inner cell mass; and
(5) culturing the isolated cell or cells in the presence of an activator of gp130 downstream signalling and an inhibitor of GSK3.

Preferably, the method comprises culturing the blastocyst in LIF, more preferably for a period of from 2 to 4 days. The isolated cell or cells are preferably cultured in serum free medium. Typically, the cells are replated as clumps. The blastocyst is also preferably cultured in serum free medium, optionally in the absence of an agonist of the BMP receptor.

It is further preferred, according to the invention, that culture of cells is carried out in an adherent culture, which may be promoted by the inclusion of a cell adhesion protein on culture substrate. It is also preferred to culture pluripotent cells according to the invention in monolayer culture, though it is optional for cells to be grown in suspension culture or as pre-cell aggregates; cells can also be grown on beads or on other suitable scaffolds such as membranes or other 3-dimensional structures.

A further component of medium for culture of pluripotent cells according to the invention, and which is preferred to be present, is a factor promoting survival and/or metabolism of the cells. In a specific embodiment of the invention, cells are cultured in the presence of insulin. An alternative factor is insulin-like growth factor and other such survival and/or metabolism promoting factors may alternatively be used.

Culture medium used in the examples of the invention preferably also comprises serum albumin. This can be used in purified or preferably recombinant form, and if in a recombinant form this has the advantage of absence of potential contaminating factors, cytokines etc. The culture medium does not need to contain serum albumin and this component can be omitted or replaced by another bulk protein or by a synthetic polymer (polyvinyl alcohol) as described by Wiles et al.

A particularly preferred medium of the invention is one that is fully defined. This medium does not contain any components which are undefined, that is to say components whose content is unknown or which may contain undefined or varying factors that are unspecified. An advantage of using a fully defined medium is that efficient and consistent protocols for culture and subsequent manipulation of pluripotent cells can be derived. Further, it is found that maintenance of cells in a pluripotent state is achievable with higher efficiency and greater predictability and that when differentiation is induced in cells cultured using a defined medium the response to the differentiation signal is more homogenous than when undefined medium is used.

The invention also provides concentrates which can be used as additives for culture medium, and kits of components, for preparation of culture medium, the resultant medium being in accordance with the invention. One kit of the invention comprises first and second containers, the first containing a gp130 agonist and the second containing a GSK3 inhibitor. The kits are preferably formulated so that the contents of each container can be added to culture medium so as to obtain a culture medium of the invention. The kits preferably contain concentrated stock solutions of their respective components.

Methods of the invention also include a method of obtaining a differentiated cell comprising culturing a pluripotent cell as described and allowing or causing the cell to differentiate, wherein the cell contains a selectable marker which is capable of differential expression in the desired differentiated cell compared with other cell-types, including pluripotent stem cells, whereby differential expression of the selectable marker results in preferential isolation and/or survival and/or division of the desired differentiated cells. The differentiated cell can be a tissue stem or progenitor cell, and may be a terminally differentiated cell.

Generally also, the invention extends to a cell obtained by following any of the methods of the invention described herein. Cells of the invention can be used in assays for drug discovery. Cells of the invention may also be used for cell therapy, and thus a method of the invention comprises using a combination of gp130 signalling and inhibition of GSK3 to derive and/or maintain pluripotent cells, deriving cells for cell therapy therefrom and using those cells in cell therapy.

Further aspects of the invention relates to culture of pluripotent cells which show reduced or absence of response to LIF, and to culture medium for such cells. In one such aspect, a method of culturing pluripotent cells comprises maintaining the cells in medium comprising an agonist of the FGF receptor and a GSK3 inhibitor. The FGF receptor agonist is preferably bFGF. The GSK3 inhibitor is preferably as described herein in relation to other aspects of the invention. The method is particularly suited to human pluripotent cells.

Another such aspect comprises expressing an Eras gene in a pluripotent cell, especially a human cell, and culturing that cell in the presence of a GSK3 inhibitor. Alternatively, GSK3 inhibitor is used to promote self renewal of a cell in which Eras are otherwise activated or in which there is an equivalent signal, e.g. expression of an Eras gene on a transgene, induction of Eras expression, overexpression of a PI3 kinase or expression of a PI3 kinase on a transgene.

Culture medium for these aspects of the invention comprises an agonist of a FGF receptor and a GSK3 inhibitor.

A number of advantages of the invention are described above or apparent. Cell culture components may be identified which are relatively non-toxic and cell permeable. The GSK3 inhibitors used in the invention can be purified easily, especially compared to, say, purification of protein cytokines. Recombinant proteins can be expensive to make and the small molecule medium components may be more cheaply produced and more stable in storage, with a wider effective concentration range.

Specific embodiments set out below used a combination of CHIR 99021 and LIF in a serum-free, fully defined medium and gave improved self renewal of mouse ES cells with very little differentiation. It is occasionally reported when culturing ES cells in the presence of BMP that there is some neurogenesis. This was not seen in the examples of the invention.

The invention is now further described in specific examples, illustrated by drawings in which:

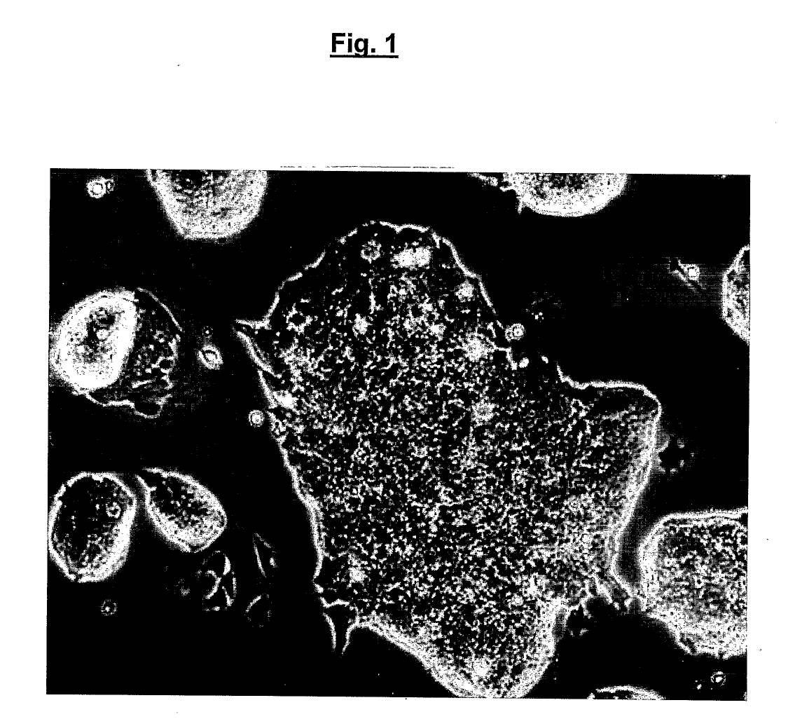

FIG. 1 shows E14.1A ES cells weaned off feeder cells and grown in LIF+CHIR99021 with serum;

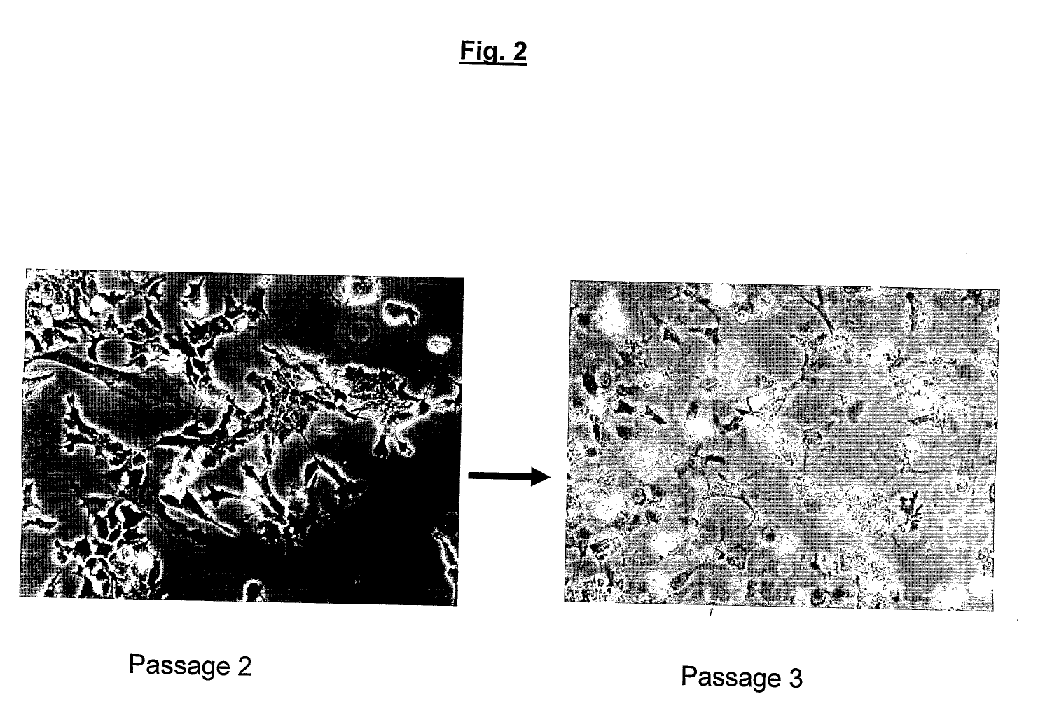

FIG. 2 shows the same ES cells in crisis in LIF and serum without CHIR99021 when they were weaned off feeder cells;

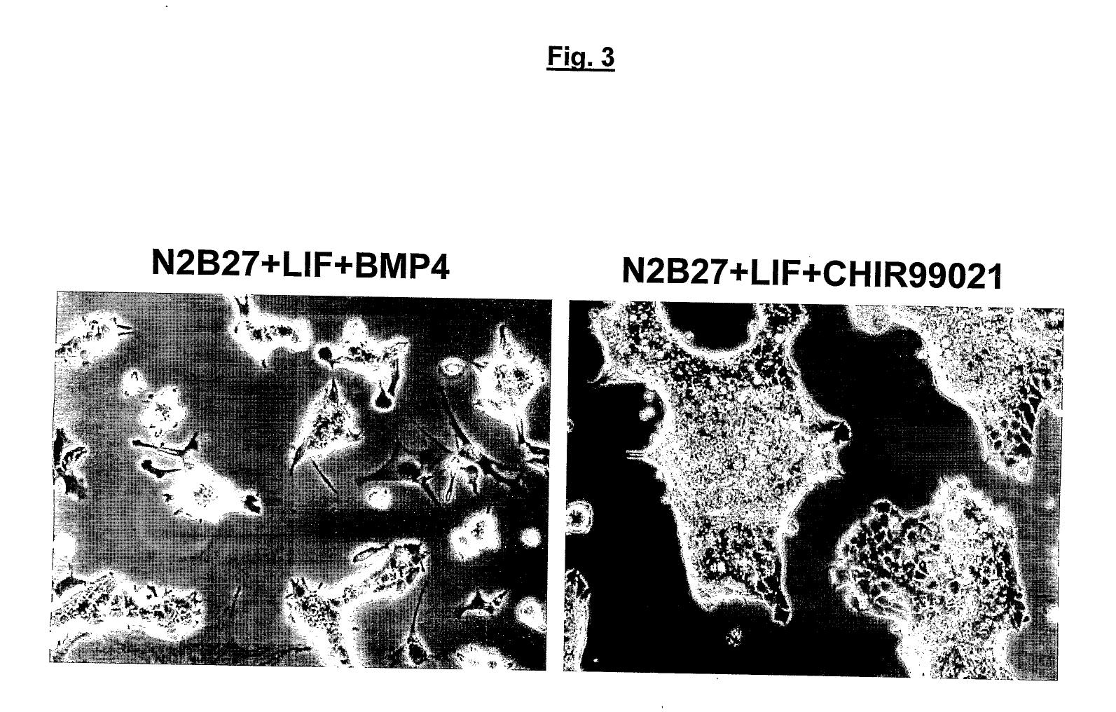

FIG. 3 shows E14.1A mouse ES cells in serum-free medium;

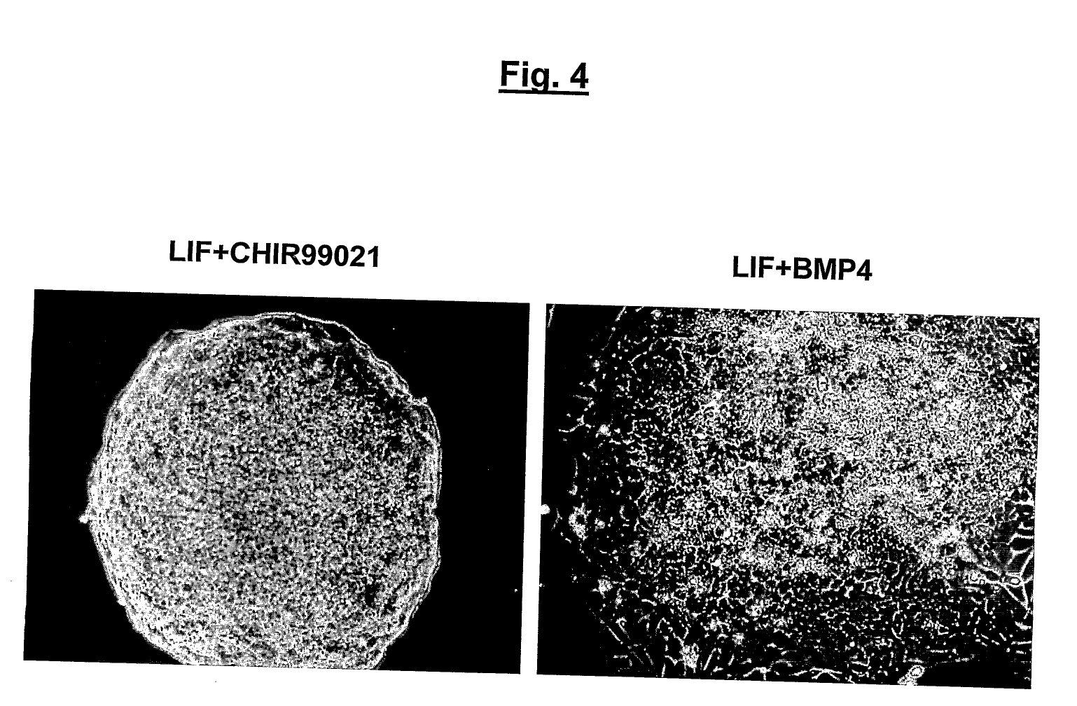

FIG. 4 shows mouse ES cells grown in defined DMEM/F12+N2 medium;

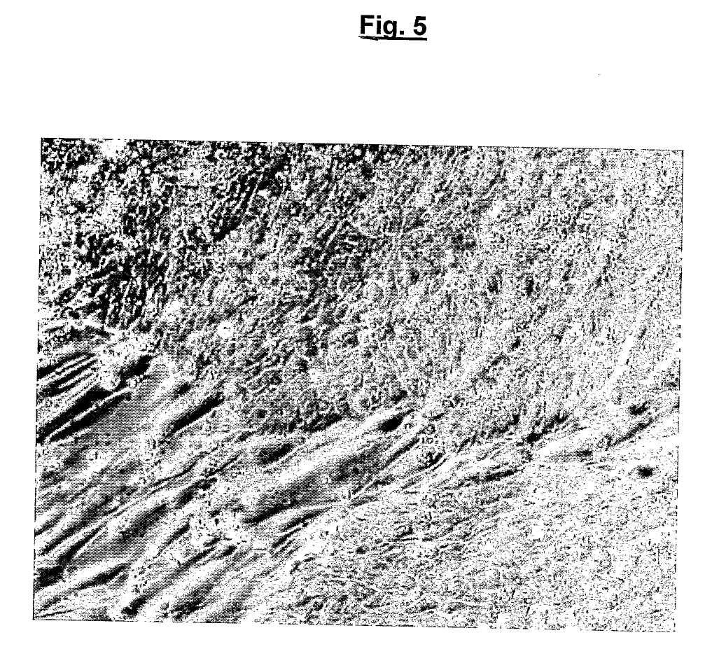

FIG. 5 shows hES181 grown in N2B27 medium with bFGF, LIF and BMP4; and

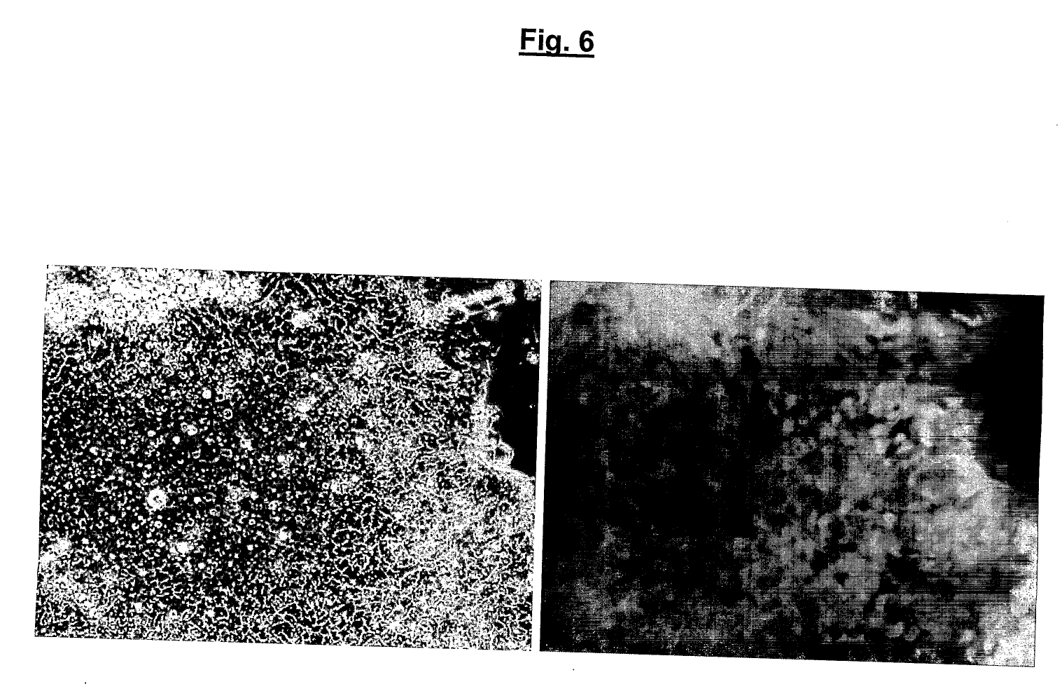

FIG. 6 shows human ES cells (hES181) stably expressing eGFP.

### EXAMPLES

### GSK-3β Inhibitors, Eras, Culture Medium and ES Cell Self-Renewal

Mouse and human ES cells were grown under various conditions, using N2B27 medium unless otherwise stated and in the presence or absence of the GSK-3β inhibitors CHIR99021, AR-AO144-18, SB216763 and SB415286.

### Preparation of N2B27 Medium:

N2 100× stock solution. For 10 ml: mix 1 ml insulin (final concentration 2.5 mg/ml) with 1 ml apo-transferrin (final concentration 10 mg/ml), 0.67 ml BSA (final concentration 5 mg/ml), 33 μl progesterone (final concentration 2 μg/ml), 100 μl putrescine (final concentration 1.6 mg/ml), 10 μl sodium selenite (final concentration 3 μM) and 7.187 ml DMEM/F12. Store at 4° C. and use within 1 month.

DMEM/F12-N2 medium: to 100 ml of DMEM/F12, add 1 ml of N2 100× stock solution. The final concentration of each component of N2 in the DMEM/F12 medium is: insulin, 25 μg/ml; apo-transferrin, 100 μg/ml; progesterone, 6 ng/ml; putrescine, 16 μg/ml; sodium selenite, 30 nM; BSA 50 μg/ml. Store at 4° C. and use within 1 month.

Neurolbasal/B27 medium: to 100 ml of Neurolbasal™ Medium, add 2 ml of B27 and 0.5-1 ml of 200 mM L-glutamine. Store at 4° C. and use within 1 month.

N2B27 medium: mix DMEM/F12-N2 medium with Neurolbasal/B27 medium in the ratio of 1:1. Add β-mercaptoethanol to a final concentration of 0.1 mM from the 0.1M stock. Store at 4° C. and use within 1 month.

### Comparative Example 1

We attempted to maintain mouse ES cells in serum-free medium containing GSK-3β inhibitors alone, i.e. without LIF, but found that this medium was not sufficient to sustain mouse ES cell self-renewal. Instead, ES cells died or differentiated in GSK-3β inhibitors alone.

### Example 1

### Mouse ES Cells

1. We found that in serum-free medium LIF plus a GSK-3, inhibitor was sufficient to sustain mouse ES cell self-renewal in both (1) N2B27 medium, and (2) fully defined medium (DMEM/F12-N2). Self renewal of ES cells was improved as ES cells grew faster in medium containing LIF plus GSK-3-β inhibitor than in medium containing LIF plus BMP4.

2. LIF plus GSK-3β inhibitors prevented feeder-dependent ES cells going into crisis when they are “weaned off” feeder cells, which ES cells were then successfully propagated in the presence of LIF plus GSK-3-β inhibitors.

### Example 2

### Human ES Cells

1. We found that conditions that could sustain human ES cell self-renewal were:

a) N2B27 medium with bFGF (10 ng/ml) and grown on feeder cells (HS27 human foreskin fibroblasts); and
b) the same as a) but adding BMP4 (5 ng/ml) and LIF (10 ng/ml) as well as bFGF.

2. Human ES cells grew in feeder-free and conditioned medium-free conditions after forced expression of an Eras gene:—

a) in N2B27 medium with bFGF, grown on laminin, fibronectin, vitronectin or matrigel;
b) the same as a) but adding BMP4 (5 ng/ml) as well as bFGF; and
c) the same as a) but adding a GSK-3β inhibitor as well as bFGF.

Thus, ES cells are maintained in a combination of a GSK3 inhibitor and gp130 signalling, or a combination of Eras signalling and GSK3 inhibitor and the invention also provides culture methods and media therefor.

1. A culture medium, comprising (a) a gp130 agonist, and (b) a GSK3 inhibitor.

2. The culture medium of claim 1, wherein the gp130 agonist is LIF, CNTF, cardiotrophin, oncostatin M, IL-6 plus sIL-6 receptor or hyper IL-6.

3. The culture medium of claim 2, wherein the gp130 agonist is (a) LIF, (b) sIL-6R and IL-6, or (c) hyper IL-6.

4. The culture medium of claim 1, wherein the GSK3 inhibitor is an inhibitor of GSK-3β.

5. The culture medium of claim 1, wherein the GSK3 inhibitor is selective for GSK3 over cdc2 and/or erk2.

6. The culture medium of claim 5, wherein the GSK3 inhibitor is at least 100 fold selective for GSK3 over cdc2.

7. The culture medium of claim 5, wherein the GSK3 inhibitor is at least 200 fold selective for GSK3 over cdc2.

8. (canceled)

9. The culture medium of claim 1, comprising N2 medium.

10. The culture medium of claim 1, comprising B27 medium.

11. A human ES culture medium according to claim 1.

12. A mouse ES cell culture medium according to claim 1.

13-16. (canceled)

17. A method of culture of pluripotent cells so as to promote self renewal, comprising maintaining the cells in medium containing:
(1) an inhibitor of GSK3; and
(2) an activator of a gp130 downstream signalling pathway.

18. The culture medium of claim 17, wherein the medium is free of serum and free of serum extract.

19. The method of claim 18, wherein the cells are mouse cells.

20. The method of claim 18, wherein the cells are human cells.

21. The method of culture of pluripotent cells of claim 17, wherein the pluripotent cells are ES cells produced by:
(1) maintaining ES cells in a pluripotent state in culture, optionally on feeders, in the presence of a cytokine acting though gp130 and serum or an extract of serum;
(2) passaging the ES cells at least once; and
(3) withdrawing the serum or the serum extract from the medium and withdrawing the feeders (if present), so that the medium is free of feeders, serum and serum extract.

22. The method of claim 21, wherein the cells are mouse cells.

23. The method of claim 21, wherein the cells are human cells.

24. A method of obtaining a transfected population of ES cells, comprising:
(1 transfecting ES cells with a construct encoding a selectable marker;
(2) plating the ES cells;
(3) culturing the ES cells in the presence of an inhibitor of GSK3 and an activator of gp130 downstream signalling pathways; and
(4) selecting for cells that express the selectable marker.

25. The method of claim 24, wherein the cells are mouse cells.

26. The method of claim 24, wherein the cells are human cells.

27. The cell culture medium of claim 1, wherein the medium is free of serum and serum extract and further comprises:
(c) basal medium; and
(d) an iron-transporter.

28. A culture medium for human pluripotent stem cells, comprising a GSK3 inhibitor, and an agonist of the FGF receptor.

29. The culture medium of claim 28, further comprising an activator of gp130 downstream signalling pathways.

30. The culture medium of claim 28, further comprising LIF, a GSK3 inhibitor, insulin, albumin and transferrin.

31. The human pluripotent stem cell medium of claim 28, further comprising (a) LIF, (b) a GSK3 inhibitor and (c) FGF.

32. The culture medium of claim 1 for culture of non-human pluripotent stem cells further comprising (a) LIF or hyper IL-6, (b) a GSK3 inhibitor and (c) an inhibitor of FGF.

33. A method of deriving a pluripotent cell from a blastocyst, comprising:
(1) culturing the blastocyst in the presence of an activator of gp130 downstream signalling, to obtain an inner cell mass;
(2) dissociating the inner cell mass;
(3) isolating a cell or cells from the dissociated inner cell mass; and
(4) culturing the isolated cell or cells in the presence of an activator of gp130 downstream signalling and an inhibitor of GSK3, thereby deriving a pluripotent cell.

34. The method of claim 33, comprising culturing the blastocyst in LIF for a period of from 2 to 4 days.

35-42. (canceled)

43. The culture medium of claim 1, wherein the GSK3 inhibitor is selected from CHIR 98014, CHIR 99021, AR-AO144-18, SB216763 and SB415286.

44. A method of expanding a stem cell population, comprising culturing the stem cells in the presence of an inhibitor of GSK3.
